(19) United States
(12) Patent Application Publication
(10) Pub. No.: US 2003/0185781 A1
(43) Pub. Date: Oct. 2, 2003

(54) Cosmetic composition forming a soft coating comprising a polymer having a non-silicone backbone and reactive functional groups

(76) Inventors: Henri Samain, Bievres (FR); Isabelle Rollat-Corvol, Paris (FR); Franck Giroud, Clichy (FR); Nathalie Mougin, Paris (FR); Aude Livoreil, Paris (FR)

Correspondence Address: Thomas L. Irving, FINNEGAN, HENDERSON, FARABOW,, GARRETT & DUNNER, L.L.P., 1300 I Street, N.W., Washington, DC, 20005-3315, US

(21) Appl. No.: 10/321,450

(22) Filed: Dec. 18, 2002

(30) Foreign Application Priority Data
Dec. 18, 2001 (FR) 01 16385

(51) Int. Cl. A61K 7/06; A61K 7/11

(52) U.S. Cl. 424/70.11; 424/70.17

(57) ABSTRACT

Nontacky cosmetic compositions, such as hair compositions, comprising at least one polymer having a non-silicone backbone, comprising at least two reactive chemical functional groups, capable of forming a soft coating on hair, a cosmetic process comprising the application of this composition to hair and its use for producing a soft coating on hair.

Disclosed herein are nontacky cosmetic compositions, such as hair compositions, comprising at least one polymer having a non-silicone backbone comprising reactive chemical functional groups, capable of forming a soft coating on hair. Also disclosed herein is a cosmetic method comprising the application of the nontacky composition to hair and its use for producing a soft coating on hair.

To determine if free organic functional groups (F) of a polymer (P) constitute reactive chemical functional groups, test 1 described below is performed:

(1) A solution or a dispersion of the polymer (P) is prepared in a cosmetically acceptable solvent chosen from water, C₁ to C₄ alcohols, esters, and ketones, such as water, such solution or dispersion having a relative polymer content ranging from 0.1 to 50% by weight, relative to the total weight of the composition;

(2) for a period ranging from 1 to 60 minutes, the solution or dispersion of polymer (P) is allowed to stand or it is subjected to at least one operation chosen from:

(i) it is stirred;

(ii) it is activated by a temperature ranging from 0° C. to 100° C.;

(iii) it is activated by a pH ranging from 1 to 13;

(iv) it is activated by at least one chemical additive (A) chosen from molecules and polymers having free chemical functional groups capable of reacting with at least one free organic functional group (F) of the polymer (P), wherein the chemical additive (A) may be, for example, a polymer having chemical functional groups identical to those of hair, chosen from amine, alcohol, carboxylic acid, disulphide and thiol functional groups;

(3) the solution or dispersion of the polymer (P) is examined by methods known to a person skilled in the art, such as by infrared or RAMAN spectrometry in order to determine if at least one of the free organic functional groups (F) of the polymer (P) has given rise to the formation of covalent bonds, which link, for example:

two atoms present in the free organic functional groups (F) belonging to different polymers (P),

one atom present in the polymer (P) and one atom present in the chemical additive (A);

(4) the polymer (P) is described as a “polymer comprising reactive functional groups” if the formation of covalent bond(s) is detected at point (3) and provided that such a covalent bond does not result exclusively from hydrolysis or oxidation thereof.

The at least one polymer having a non-silicone backbone, comprising at least two reactive functional groups as disclosed herein is capable of forming covalent bonds by carrying out the test 1 as described above. This characteristic distinguishes the at least one polymer disclosed herein from most known polymers in the field of hair compositions, which do not react, under the conditions of the test 1, by forming strong bonds, but at the very most by interacting with each other or with additives through bonds of the hydrogen or saline bond type.

As disclosed herein, the expression “polymer having a non-silicone backbone” is understood to mean a polymer which does not exclusively consist of a succession of —Si—O—Si— in its main chain.

The at least one polymer disclosed herein excludes polymers having photoactivable reactive functional groups, that is to say polymers containing chemical functional groups which, when irradiated at a wavelength ranging from 200 to 800 nm, give rise, in at least one step, to the formation of new covalent bonds.

Cosmetic products intended for treating hair often use polymers. They make it possible to obtain, for example, effects of retaining the shape of the hairstyle, effects of softness or effects of gloss.

Some compositions using polymers can have disadvantages which may be inconvenient. For example, if, after having applied a product containing polymers, a person passes their hand through their hair, some of the polymers may, at the time of contact, become deposited on the fingers. This phenomenon of transfer, although it is partial, can leave an impression of dirty or sticky hair. The extent of this transfer may depend on climatic conditions. Thus, for example, in humid conditions, it is often considerable.

Moreover, when sebum covers the hair, either along its length, or at the root, and when a cosmetic product, such as a hairstyling product, is applied thereto, the product may not only be ineffective, but may moreover make the hair even more artificially glossy and more dirty.

Another disadvantage of the polymers commonly used in cosmetics lies in the fact that they sometimes make the hair dry, which may cause impairment of its feel and a decrease in the expected effect of the product, for example, the effect of fixing and retaining the hairstyle. An additional disadvantage is that the polymers deposited on the hair can be very quickly removed during shampooing.

For example, when it is chosen to use polymers which form a coating, such as a film, of soft consistency, on the hair, the feel of the hair may often be particularly rough or sticky, and unpleasant. In addition, this soft coating can be immediately removed during washing of the hair, and it may therefore be necessary to reapply the product, at least after each shampoo wash.

A need therefore exists for producing nontacky cosmetic compositions which are improved compared with prior art compositions, and, for example, compositions that do not stick to the fingers during application to hair, do not cause the hair to dry out, and can give at least one good cosmetic properties to the hair, even in the presence of sebum, and can be remanent with respect to repeated washings.

Surprisingly and unexpectedly, the inventors have discovered that it is possible to achieve at least one of the objectives listed above by selecting the polymers introduced into the nontacky cosmetic compositions, according to the nature of the chemical functional groups which they carry and according to the characteristics of the film which they form on the hair.

Therefore, disclosed herein is a nontacky cosmetic composition, such as a hair composition, comprising, in a cosmetically acceptable medium, at least one polymer having a non-silicone backbone, comprising at least two nonphotoactivable, reactive chemical functional groups, which may be identical or different, characterized in that:

(i) a film obtained by drying the composition at room temperature (22±2° C.) and at a relative humidity level of 50%±5%, has a Young's modulus ranging from 1 to 100 MPa, measured at a thickness of 0.5 mm and at a pulling rate of 20 mm/min,

(ii) the at least two reactive chemical functional groups are chosen from the following monovalent and divalent groups:

epoxy,

anhydride,

acid chloride,

ethyleneimino,

aldehyde,

acetal and hemiacetal,

aminal and hemiaminal,

ketone, alpha-haloketone and alpha-hydroxyketone,

lactone and thiolactone,

isocyanate,

thiocyanate,

N-hydroxysuccinimide ester,

imide,

imine,

imidate,

oxazoline, oxazolinium, oxazine and oxazinium,

pyridylthiol,

thiosulphate,

acetoalkylate corresponding to the formula:

—OCO—A′—COCH₃,

wherein A′ is a group chosen from a bond and linear and branched alkylene groups comprising from 1 to 5 carbon atoms,

AX, and

ASO₂X,

wherein A is chosen from alkylene, arylene and aralkylene groups comprising from 1 to 22 carbon atoms, which may be optionally interrupted by at least one unsaturated ring, and may optionally comprise at least one hetero atom, such as N, S and O. X is a leaving group chosen from halogens, OSO₃H, OSO₂CH3, OSO₂C₂H₅, OSO₂Tos, N(CH3)₃, OPO₃R₂, and CN,

wherein Tos is a tosylate group,

and R is chosen from a hydrogen atom and C₁ to C₅ alkyl radicals.

Tacky compositions are compositions which give, after application to keratin fibers and drying, a hairstyling material having a peeling profile defined by at least:

a maximum peeling force Fₘₐₓ>1 Newton, and

for example, in addition, an energy of separation E_{s(M/G)} of the material brought into contact with a glass surface of less than 300 μJ,

wherein Fₘₐₓ and E_{s(M/G)} being measured according to the protocol described in international application WO 98/38969 are excluded from the present application.

Another embodiment disclosed herein relates to a cosmetic method comprising the application of this nontacky composition.

Yet another embodiment relates to the use of this nontacky composition for forming a soft coating on hair.

As disclosed herein, the expression “coating” means an envelope formed at the surface of each hair, after drying the nontacky cosmetic composition. This envelope has virtually the shape of a hollow cylinder which may extend from the root to the tip of the hair and which adheres strongly thereto.

Without being bound by any theory, the inventors believe that the polymers having a non-silicone backbone present in the nontacky cosmetic compositions disclosed herein may, because of their identical or different reactive functional groups, react completely or partially, with themselves, with each other, with sensitized or nonsensitized hair, and/or with at least one reactive constituent of the hair composition, occurring after application of the cosmetic composition to the hair, in order to form a coating. The mechanism of formation of the coating may be better understood by means of the examples of reaction schemes below:

1) Reaction of two polymers comprising reactive epoxy functional groups with a reactive ingredient of the composition having the formula RHN—A—NHR′,

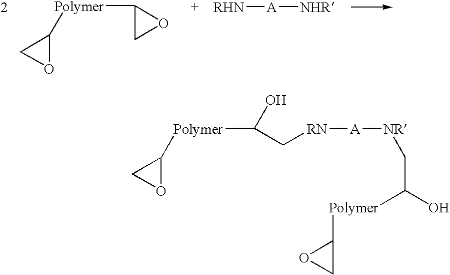

2) Reaction of a polymer comprising two reactive epoxy functional groups with an amine functional group of hair,

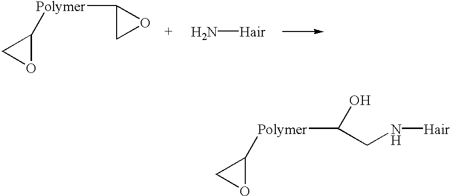

As described herein, the reaction of the polymers having a non-silicone backbone with each other and with the hair, for example, may be promoted by providing heat, by adding constituents, for example, pH regulating agents, chemical active agents, such as oxidizing agents, reducing agents, polymerization inhibitors and polymerization catalysts.

In one embodiment, the polymer having a non-silicone backbone, comprising at least two reactive functional groups, comprises less than 50%, in numeric terms, of carboxylic acid ester functional groups, relative to the total number of reactive chemical functional groups.

In another embodiment, the leaving group X is a halogen chosen from bromine, chlorine, iodine and fluorine.

When the polymer having a non-silicone backbone, comprising at least two reactive functional groups, comprises at least one epoxy group, the at least one epoxy group is, for example, monovalent and is chosen from groups corresponding to formula (I):

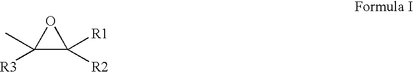

wherein R1, R2 and R3, which may be identical or different, are each chosen from:

a hydrogen atom,

linear and branched alkyl groups comprising from 1 to 20 carbon atoms, which may be optionally interrupted by at least one hetero atom chosen from O, N, S, Si and F, and may be optionally substituted with at least one radical chosen from hydroxyl and amino radicals,

aryl groups comprising from 6 to 22 carbon atoms,

aralkyl groups, wherein the alkyl groups comprise from 1 to 20 carbon atoms, and

5- to 7-membered heterocycles.

When the polymer having a non-silicone backbone, comprising at least two reactive functional groups comprises at least one anhydride group of a carboxylic acid, the at least one anhydride group of a carboxylic acid is, for example, monovalent and is chosen from:

(a) groups corresponding to formula (II):

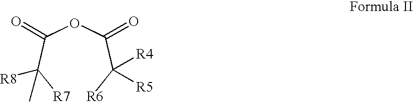

wherein, R4, R5, R6, R7 and R8, which may be identical or different, have the same meanings as those given for R1, R2 and R3 in the formula (I), and

(b) groups corresponding to formula (III):

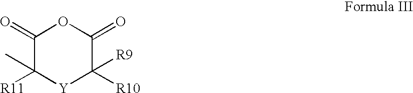

wherein Y is chosen from:

a bond,

hetero atoms chosen from O, N, S, Si and F,

alkyl and alkylene radicals, that can be unsubstituted or substituted with at least one radical chosen from hydroxyl and amino radicals, comprising from 1 to 5 carbon atoms,

aralkylene radicals comprising from 7 to 10 carbon atoms, and

polydimethylsiloxane radicals comprising from 1 to 6 silicon atoms, and

R9, R10 and R11, which may be identical or different, have the same meanings as those given for R1, R2 and R3 in the formula (I).

When the polymer having a non-silicone backbone, comprising at least two reactive functional groups, comprises at least one acetoalkylate group, the acetoalkylate group is, for example, included in the group corresponding to formula (IV):

—R′₁—OCO—A′—COCH₃ Formula IV

wherein R′₁ is obtained by eliminating a hydrogen atom of the radical R1 as defined in the formula (I) and wherein A′ has the meaning given above.

When the polymer having a non-silicone backbone, comprising at least two reactive functional groups, comprises at least one acid chloride group, the acid chloride group, for example, is included in a group corresponding to formula (V):

—R′₁—COCl Formula V

wherein R′₁ has the same meaning as in the formula (IV).

When the polymer having a non-silicone backbone, comprising at least two reactive functional groups comprises at least one isocyanate group, the isocyanate group is, for example, included in a group corresponding to formula (VI):

—R′₁—NCO Formula VI

in which R′₁ has the same meaning as in the formula (IV).

When the polymer having a non-silicone backbone, comprising at least two reactive functional groups, comprises at least one acetal group, the acetal group is, for example, monovalent and is included in at least one of the formulae (VII), (VIII) and (IX):

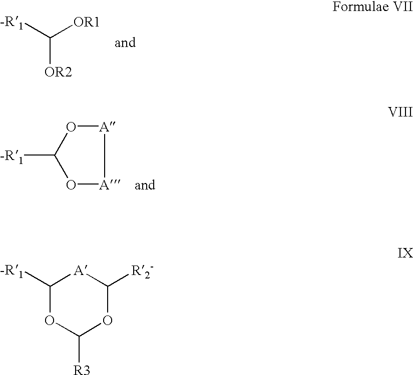

wherein:

R1, R2 and R3, which may be identical or different, have the same meanings as defined in the formula (I),

R′₁ and R′₂ are obtained by eliminating a hydrogen atom of the radical R1 or R2 as defined in the formula (I),

A′ has the same meaning as defined above,

A″ and A′″, which may be identical or different, are each chosen from linear and branched alkyl and alkylene groups comprising from 1 to 5 carbon atoms, which may be optionally interrupted by at least one hetero atom chosen from O, N, S, Si and F, and may be optionally substituted with at least one radical chosen from hydroxyl and amino radicals.

For example, the at least one polymer having a non-silicone backbone comprising at least two reactive functional groups is chosen from:

(a) copolymers synthesized from (meth)acrylate and acrylate monomers comprising acetoacetate functional groups, corresponding to the general formula (IV.1):

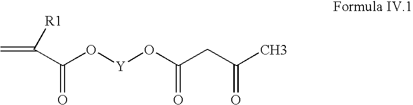

wherein R1 is chosen from H and CH₃, and

Y has the same meaning as defined in the formula (III).

In one embodiment, wherein R1 is CH₃, and Y is —(CH₂)₂—.

(b) polymers synthesized from (meth)acrylate and (meth)acrylamide monomers comprising acetal functional groups, such monomers chosen from those of formulae (VII.1) and (VIII.1):

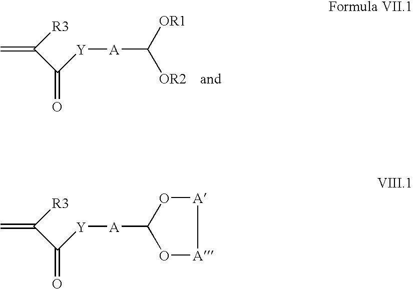

wherein Y is chosen from O and NH,

R3 is chosen from H and CH₃, and

A, A″, A′″, R1 and R2 have the same meanings as defined above.

Copolymers synthesized from N-ethyl acetal acrylamide of formula (VIII.2)

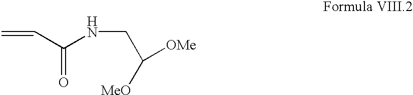

can also be used.

(c) copolymers comprising acetal functional groups obtained by chemical modification of polymers chosen from synthetic and natural polymers, such as copolymers derived from the reaction of at least one aldehyde with the poly(vinyl alcohol/vinyl acetate) of general formula (X):

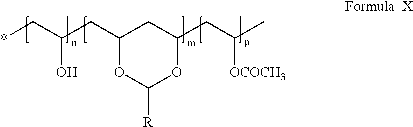

wherein R has the same meaning as defined above,

n, m and p, which may be identical or different, each range from 1 to 10 000.

Such syntheses are known to a person skilled in the art and are described in le Précis de Matières Plastiques, J. P. Trotigon, J. Verdu, Editions Nathan, 1996.

The polymers having a non-silicone backbone as disclosed herein may be obtained according to conventional methods of polymerization or of modification of polymers.

To obtain such polymers, the production process may comprise, for example, at least one of the following operations:

a polycondensation,

an opening of at least one ring chosen from rings comprising from 2 to 9 carbon atoms and rings comprising from 2 to 4 silicon atoms, wherein the at least one ring may comprise at least one hetero atom chosen from N, O, S and Si;

a polymerization of unsaturated monomers, chosen from free-radical and ionic polymerizations, by group transfer.

The polymeric non-silicone backbone as disclosed herein may be linear, branched, hyperbranched or dendritic. It may comprise at least one type of repeating unit, and may therefore be chosen from homopolymers and copolymers which can be random, alternating and block.

As disclosed herein, the at least two reactive functional groups are distributed along either the main or secondary chains of the at least one polymer having a non-silicone backbone, and may be optionally at the ends of the chains in the case of branched, hyperbranched and dendritic polymers.

When the polymer having a non-silicone backbone as disclosed herein is formed by a polymerization method as described above, the at least two reactive functional groups may be present on the monomers serving as starting material for the polymerization, or may be formed by reaction of the monomers with each other during the polymerization, or may be provided by at least one chemical operation in addition to the polymerization, for example, an operation comprising grafting, such as on to the polymer obtained, molecular or polymeric units comprising the appropriate reactive functional groups chosen from those of formulae (I) to (IX).

To perform a polycondensation, the operating protocols described in Step Polymerisation” in Principles of Polymerization, G. ODIAN, 3 ed., Wiley Interscience, may, for example, be followed.

In the case of a polycondensation, the monomers used as starting material are, for example, chosen from diamines and diols reacting with diisocyanates, diacids, and diesters, and which lead to polyurethanes, polyamides, polyesters, and aziridines and derivatives thereof leading to polyalkyleneimines, such as polyethyleneimines and derivatives thereof.

For example, a polyurethane may be obtained by reaction of the following monomers: isophorone diisocyanate, hexamethylene diisocyanate, methylenebiscyclohexane diisocyanate, and polytetramethylene glycol dihydroxyl.

To perform an opening of at least one ring chosen from rings comprising from 2 to 9 carbon atoms and rings comprising from 2 to 4 silicon atoms, wherein the at least one ring optionally comprises at least one hetero atom chosen from N, O, S, and Si, the procedures described in “Ring Opening Polymerization” in Comprehensive Polymer Science, Perg. Press, vol. 3, may, for example, be followed.

In the case of a ring-opening operation, the monomers used as the starting material to form the polymers are, for example, chosen from cyclic esters (lactones) and cyclic amides (lactams), such as:

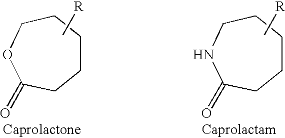

wherein R has the same meaning as defined above.

In the case where the polymer having a non-silicone backbone as disclosed herein is formed by a production process comprising a ring-opening operation, the reactive functional groups may be present in the monomers serving as the starting material and comprising a ring, for example, as chemical substituents present on the rings, or may be formed after the mutual reaction of these monomers comprising a ring, or may be provided by at least one chemical operation in addition to the ring-opening operation, for example, a separate operation comprising grafting molecular or polymeric units comprising the appropriate reactive functional groups chosen from those of the formulae (I) to (IX).

To perform a free-radical or anionic polymerization, the procedures described in “Radical Polymerization and Anionic Polymerization” in Principles of Polymerization, G. ODIAN, 3 ed., Wiley Interscience, may, for example, be followed.

In the case of a free-radical or anionic polymerization, the monomers used as starting material, for forming the polymers, are, for example, chosen from vinyls, dienes, (meth)acrylates, and (meth)acrylamides.

In the case of a free-radical or anionic polymerization, the polymer comprises at least ten units linked by covalent bonds. The at least two reactive functional groups present on the polymer entering into the constitution of the nontacky compositions as disclosed herein, may be already present on the monomers serving as the starting material for the free-radical reaction, or may be optionally formed during the free-radical reaction, or alternatively may, for example, be provided on the polymer by any additional chemical operation.

It is also possible to use natural polymers and natural polymers that are chemically modified to include the reactive functional groups listed above. Mention may be made, for example and without limitation, of polysaccharides (cellulose, chitosan, guar and derivatives thereof), and polypeptides (polyaspartic acid, polylysine and derivatives thereof). As described herein, such polymers having a non-silicone backbone may comprise, naturally or after modification, reactive functional groups chosen from hydroxyl, amine, carboxylic acid, thiol, aldehyde and epoxy functional groups, the reactivity of which is used without further modification in the nontacky composition (for example, with polymers bearing epoxy functional groups) or to provide the chemical functional groups listed above.

By way of non-limiting example, the polymer may be modified as follows:

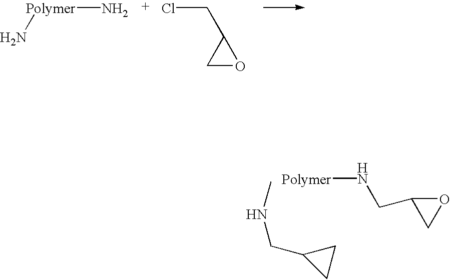

In order to determine the Young's modulus, a composition comprising at least one polymer as disclosed herein is used in an appropriate quantity in order to obtain, in a teflon matrix, a dry film having a thickness of 500±50 μm. The drying is continued until the weight of the film no longer changes.

To measure the Young's modulus, a pulling test is carried out. The film is cut into test pieces of rectangular shape, 80 mm in length and 15 mm in width.

The tests are carried out on an apparatus marketed under the name Lloyd or marketed under the name Zwick under the same temperature and humidity conditions as for the drying, i.e., a temperature of 22±2° C. and a relative humidity level of 50±5%.

The test pieces are pulled at the speed of 20 mm/min and the distance between the jaws is 50±1 mm.

Another embodiment as disclosed herein relates to a cosmetic process comprising applying to hair a nontacky cosmetic composition as disclosed herein.

In another embodiment as disclosed herein, the process comprises at least one additional operation chosen from causing a modification of pH, a rise in temperature, adding at least one additive, and rinsing.

In one embodiment, before applying the nontacky composition as disclosed herein, at least one composition chosen from care, coloring, permanent waving, hair makeup, hairstyle fixing and hairstyle retaining compositions is applied to hair.

In the nontacky compositions as disclosed herein, the at least one polymer having a non-silicone backbone comprising at least two reactive functional groups, is present in a concentration ranging from 0.05 to 20% by weight, such as from 0.1 to 15% by weight, and further such as from 0.25 to 10% by weight, relative to the total weight of the composition.

As disclosed herein, the composition, for example, further comprises at least one conventional cosmetic additive chosen from fixing polymers, thickeners, anionic, nonionic, cationic and amphoteric surfactants, perfumes, preservatives, sunscreens, proteins, vitamins, provitamins, anionic, nonionic, cationic and amphoteric nonfixing polymers, mineral, vegetable and synthetic oils, ceramides, pseudoceramides, modified and nonmodified, linear and cyclic, volatile and nonvolatile silicones, pH-regulating agents, oxidizing agents, reducing agents, inhibitors, catalysts and any other additives conventionally used in cosmetic compositions intended to be applied to hair.

The cosmetically acceptable medium is chosen from water, at least one cosmetically acceptable solvent, such as alcohols, esters, ketones, and volatile cyclic silicones, and water-solvent mixtures. For example, the at least one cosmetically acceptable solvent may be chosen from C₁-C₄ alcohols.

In one embodiment as disclosed herein, the nontacky composition is packaged in an aerosol device, wherein the nontacky composition further comprises at least one propellant which may be chosen from volatile hydrocarbons, such as n-butane, propane, isobutane, pentane, and halogenated hydrocarbons. Carbon dioxide gas, nitrous oxide, dimethyl ether (DME), nitrogen and compressed air may also be used as the at least one propellant. It is also possible to use mixtures of propellants. For example, dimethyl ether can be used.

The at least one propellant is present, for example, at a concentration ranging from 5 to 90% by weight, and further, for example, from 10 to 60% by weight, relative to the total weight of the composition in the aerosol device.

The nontacky compositions as disclosed herein may be applied to dry or wet hair.

The invention will be more fully illustrated with the aid of the following non-limiting example.

### EXAMPLE

1. Preparation of the polymer P1: adipic acid/diethylenetriamine/piperazine/epichlorohydrin polycondensate

The operating procedure for the production of the polymer P1 was taken from FR 2 252 840.

The constitution of the polymer prepared before crosslinking with epichlorohydrin may be represented by the two units below randomly distributed in the proportion of 2:1

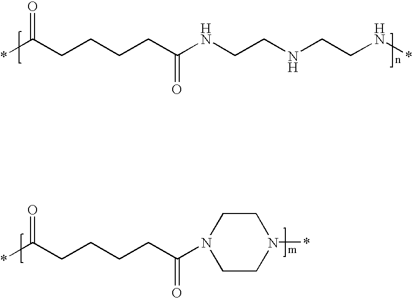

The mixture of 438 g (3 mol) of adipic acid and 86 g (1 mol) of piperazine was heated, with stirring and under a nitrogen atmosphere, for two hours at a temperature ranging from 120 to 135° C. 206 g (2 mol) of diethylenetriamine were then added at this temperature and over 90 minutes. The water formed was distilled off for one hour at a temperature ranging from 140 to 170° C. at ordinary pressure, and then for 1 hour at a temperature ranging from 170 to 175° C. at 15 mmHg.

The product thus obtained was present in the form of a brittle hard resin which was transparent and green-yellow in color.

72 g of epichlorohydrin were added at room temperature, with stirring, to 200 g of this resin dissolved in 800 g of water. The mixture was then heated to 90° C. and 8 g of epichlorohydrin were further added in small fractions at intervals ranging from 5 to 10 minutes. The solution was then rapidly diluted with 1090 g of water in order to obtain a concentration of 10%.

The quantity of crosslinking agent used was stoichiometric relative to the amine groups of the polyamido amine, which ensured the presence of reactive epoxy functional groups in the molecules of the crosslinked polymer.

2. The following composition was prepared:

| | | |
|---|---|---|
| Polymer 1 | 5 | g |
| Monoethanolamine | 1 | g |
| Water | qs 100 | g |

The Young's modulus of the film obtained from the composition was about 6.3 Mpa.

The composition, when applied to hair and dried, conferred on it a soft coating which was resistant to shampooing.

What is claimed is:

1. A nontacky cosmetic composition comprising, in a cosmetically acceptable medium, at least one polymer having a non-silicone backbone, comprising at least two nonphotoactivable, reactive chemical functional groups, which may be identical or different, wherein:
(i) a film obtained by drying the composition at room temperature (22±2° C.) and at a relative humidity level of 50%±5%, has a Young's modulus ranging from 1 to 100 MPa, measured at a thickness of 0.5 mm and at a pulling rate of 20 mm/min;
(ii) the at least two nonphotoactivable reactive chemical functional groups are chosen from the following monovalent and divalent groups:
epoxy,
anhydride,
acid chloride,
ethyleneimino,
aldehyde,
acetal and hemiacetal,
aminal and hemiaminal,
ketone, alpha-haloketone and alpha-hydroxyketone,
lactone and thiolactone,
isocyanate,
thiocyanate,
N-hydroxysuccinimide ester,
imide,
imine,
imidate,
oxazoline, oxazolinium, oxazine and oxazinium,
pyridylthiol,
thiosulphate,
acetoalkylate corresponding to the formula:
—OCO—A′—COCH₃,
wherein A′ is chosen from a bond and linear and branched alkylene groups comprising from 1 to 5 carbon atoms,
AX, and
ASO₂X,
wherein A is a group chosen from alkylene, arylene and aralkylene groups comprising from 1 to 22 carbon atoms, which may be optionally interrupted by at least one unsaturated ring, and may optionally comprise at least one hetero atom, and
X is a leaving group chosen from halogens, OSO₃H, OSO₂CH3, OSO₂C₂H₅, OSO₂Tos, N(CH3)₃, OPO₃R₂, and CN,
wherein Tos is a tosylate group, and
R is chosen from a hydrogen atom and C₁ to C₅ alkyl radicals.

2. The composition according to claim 1, wherein the composition is a hair composition.

3. The composition according to claim 1, wherein the at least one hetero atom in the definition of A is chosen from N, S, and O.

4. The composition according to claim 1, wherein the polymer having a non-silicone backbone comprising at least two reactive chemical functional groups comprises less than 50% in numerical terms of carboxylic acid ester functional groups, relative to the total number of reactive chemical functional groups.

5. The composition according to claim 1, wherein X is a halogen chosen from bromine, chlorine, iodine and fluorine.

6. The composition according to claim 1, wherein, in (ii), the epoxy group is monovalent and is chosen from groups corresponding to formula (I): 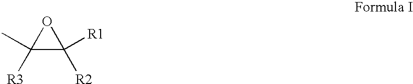
wherein R1, R2 and R3, which may be identical or different, are each chosen from:
a hydrogen atom,
linear and branched alkyl groups comprising from 1 to 20 carbon atoms, which may be optionally interrupted by at least one hetero atom chosen from O, N, S, Si and F, and may be optionally substituted with at least one radical chosen from hydroxyl and amino radicals,
aryl groups comprising from 6 to 22 carbon atoms,
aralkyl groups, wherein the alkyl group comprises from 1 to 20 carbon atoms, and
5- to 7-membered heterocycles.

7. The composition according to claim 1, wherein, in (ii), the anhydride group is chosen from carboxylic acid anhydride groups.

8. The composition according to claim 7, wherein the carboxylic acid anhydride groups are monovalent and are chosen from groups corresponding to formula (II): 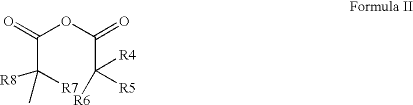
wherein R4, R5, R6, R7 and R8, which may be identical or different, are each chosen from:
a hydrogen atom,
linear and branched alkyl groups comprising from 1 to 20 carbon atoms, which may be optionally interrupted by at least one hetero atom chosen from O, N, S, Si and F, and may be optionally substituted with at least one radical chosen from hydroxyl and amino radicals,
aryl groups comprising from 6 to 22 carbon atoms,
aralkyl groups, wherein the alkyl group comprises from 1 to 20 carbon atoms, and
5- to 7-membered heterocycles.

9. The composition according to claim 7, wherein the carboxylic acid anhydride groups are monovalent and are chosen from groups corresponding to formula (III): 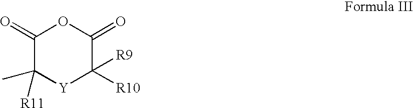
wherein Y is chosen from:
a bond,
hetero atoms chosen from O, N, S, Si and F,
alkyl and alkylene radicals that are unsubstituted or substituted with at least one radical chosen from hydroxyl and amino radicals, comprising from 1 to 5 carbon atoms,
aralkylene radicals comprising from 7 to 10 carbon atoms, and
polydimethylsiloxane radicals comprising from 1 to 6 silicon atoms, and
R9, R10 and R11, which may be identical or different, are each chosen from:
a hydrogen atom,
linear and branched alkyl groups comprising from 1 to 20 carbon atoms, which may be optionally interrupted by at least one hetero atom chosen from O, N, S, Si and F, and may be optionally substituted with at least one radical chosen from hydroxyl and amino radicals,
aryl groups comprising from 6 to 22 carbon atoms,
aralkyl groups, wherein the alkyl group comprises from 1 to 20 carbon atoms, and
5- to 7-membered heterocycles.

10. The composition according to claim 1, wherein, in (ii), the acetoalkylate group is included in a group corresponding to formula (IV):
—R′₁—OCO—A′—COCH₃ Formula IV
wherein R′₁ is obtained by eliminating a hydrogen atom of radical R1, wherein the radical R1 is chosen from:
a hydrogen atom,
linear and branched alkyl groups comprising from 1 to 20 carbon atoms, which may be optionally interrupted by at least one hetero atom chosen from O, N, S, Si and F, and may be optionally substituted with at least one radical chosen from hydroxyl and amino radicals,
aryl groups comprising from 6 to 22 carbon atoms,
aralkyl groups, wherein the alkyl group comprises from 1 to 20 carbon atoms, and
5- to 7-membered heterocycles; and
A′ is chosen from a bond and linear and branched alkylene groups comprising from 1 to 5 carbon atoms.

11. The composition according to claim 1, wherein, in (ii), the acid chloride group is included in a group corresponding to formula (V):
—R′₁—COCl Formula V
wherein R′₁ is obtained by eliminating a hydrogen atom of radical R1, wherein the radical R1 is chosen from:
a hydrogen atom,
linear and branched alkyl groups comprising from 1 to 20 carbon atoms, which may be optionally interrupted by at least one hetero atom chosen from O, N, S, Si and F, and may be optionally substituted with at least one radical chosen from hydroxyl and amino radicals,
aryl groups comprising from 6 to 22 carbon atoms,
aralkyl groups, wherein the alkyl group comprises from 1 to 20 carbon atoms, and
5- to 7-membered heterocycles.

12. The composition according to claim 1, wherein, in (ii), the isocyanate group is included in a group corresponding to formula (VI):
—R′₁—NCO Formula VI
wherein R′₁ is obtained by eliminating a hydrogen atom of radical R1, wherein the radical R1 is chosen from:
a hydrogen atom,
linear and branched alkyl groups comprising from 1 to 20 carbon atoms, which may be optionally interrupted by at least one hetero atom chosen from O, N, S, Si and F, and may be optionally substituted with at least one radical chosen from hydroxyl and amino radicals,
aryl groups comprising from 6 to 22 carbon atoms,
aralkyl groups, wherein the alkyl group comprises from 1 to 20 carbon atoms, and
5- to 7-membered heterocycles.

13. The composition according to claim 1, wherein, in (ii), the acetal group is monovalent and is included in at least one of the formulae (VII), (VIII) and (IX): 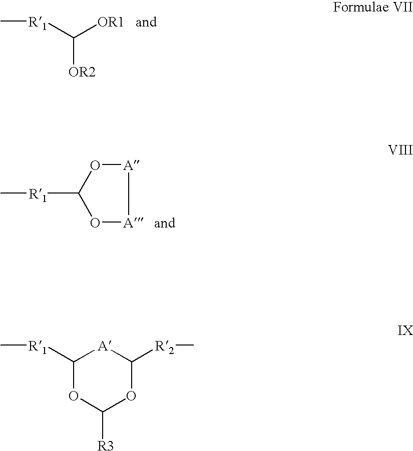
wherein:
R1, R2 and R3, which may be identical or different, are each chosen from:
a hydrogen atom,
linear and branched alkyl groups comprising from 1 to 20 carbon atoms, which may be optionally interrupted by at least one hetero atom chosen from O, N, S, Si and F, and may be optionally substituted with at least one radical chosen from hydroxyl and amino radicals,
aryl groups comprising from 6 to 22 carbon atoms,
aralkyl groups, wherein the alkyl group comprises from 1 to 20 carbon atoms, and
5- to 7-membered heterocycles;
R′₁ and R′₂ are obtained by eliminating a hydrogen atom of the radicals R₁ and R₂;
A′ is chosen from a bond, and linear and branched alkylene groups comprising from 1 to 5 carbon atoms;
A′ and A′″, which may be identical or different, are each chosen from linear and branched alkyl and alkylene groups comprising from 1 to 5 carbon atoms, which may be optionally interrupted by at least one hetero atom chosen from O, N, S, Si and F, and may be optionally substituted with at least one radical chosen from hydroxyl and amino radicals.

14. The composition according to claim 1, wherein the at least one polymer having a non-silicone backbone, comprising at least two reactive chemical functional groups is obtained by a process comprising at least one of the following operations:
a polycondensation,
an opening of at least one ring chosen from rings comprising from 2 to 9 carbon atoms and rings comprising from 2 to 4 silicon atoms wherein the at least one ring may comprise at least one hetero atom; and
a polymerization of unsaturated monomers, chosen from free-radical and ionic polymerizations, by group transfer.

15. The composition according to claim 14, wherein the at least one hetero atom is chosen from N, O, S, and Si.

16. The composition according to claim 1, wherein the at least one polymer having a non-silicone backbone comprising at least two reactive chemical functional groups is present in the composition in a concentration ranging from 0.05 to 20% by weight, relative to the total weight of the composition.

17. The composition according to claim 16, wherein the at least one polymer having a non-silicone backbone, comprising at least two reactive chemical functional groups is present in the composition in a concentration ranging from 0.1 to 15% by weight, relative to the total weight of the composition.

18. The composition according to claim 17, wherein the at least one polymer having a non-silicone backbone, comprising at least two reactive chemical functional groups is present in the composition in a concentration ranging from 0.25 to 10% by weight, relative to the total weight of the composition.

19. The composition according to claim 1, further comprising at least one cosmetic additive chosen from fixing polymers; thickeners; anionic, nonionic, cationic and amphoteric surfactants; perfumes; preservatives; sunscreens; proteins; vitamins; provitamins; anionic, nonionic, cationic and amphoteric nonfixing polymers; mineral, vegetable and synthetic oils; ceramides; pseudoceramides; modified and nonmodified, linear and cyclic, volatile and nonvolatile silicones; pH-regulating agents; oxidizing agents; reducing agents; inhibitors; and catalysts.

20. The composition according to claim 1, wherein the cosmetically acceptable medium is chosen from water, at least one cosmetically acceptable solvent, and mixtures thereof.

21. The composition according to claim 20, wherein the at least one cosmetically acceptable solvent is chosen from alcohols and volatile cyclic silicones.

22. The composition according to claim 21, wherein the alcohols are chosen from C₁-C₄ alcohols.

23. An aerosol device comprising at least one propellant and a composition comprising, in a cosmetically acceptable medium, at least one polymer having a non-silicone backbone, comprising at least two nonphotoactivable reactive chemical functional groups, which may be identical or different, wherein:
(i) a film obtained by drying the composition at room temperature (22±2° C.) and at a relative humidity level of 50%±5%, has a Young's modulus ranging from 1 to 100 MPa, measured at a thickness of 0.5 mm and at a pulling rate of 20 mm/min;
(ii) the at least two nonphotoactivable reactive chemical functional groups are chosen from the following monovalent and divalent groups:
epoxy,
anhydride,
acid chloride,
ethyleneimino,
aldehyde,
acetal and hemiacetal,
aminal and hemiaminal,
ketone, alpha-haloketone and alpha-hydroxyketone,
lactone and thiolactone,
isocyanate,
thiocyanate,
N-hydroxysuccinimide ester,
imide,
imine,
imidate,
oxazoline, oxazolinium, oxazine and oxazinium,
pyridylthiol,
thiosulphate,
acetoalkylate corresponding to the formula:
—OCO—A′—COCH₃,
wherein A′ is chosen from a bond and linear and branched alkylene groups comprising from 1 to 5 carbon atoms,
AX, and
ASO₂X,
wherein A is a group chosen from alkylene, arylene and aralkylene groups comprising from 1 to 22 carbon atoms, which may be optionally interrupted by at least one unsaturated ring, and may optionally comprise at least one hetero atom,
X is a leaving group chosen from halogens, OSO₃H, OSO₂CH3, OSO₂C₂H₅, OSO₂Tos, N(CH3)₃, OPO₃R₂, and CN,
wherein Tos is a tosylate group, and
R is chosen from a hydrogen atom and C₁ to C₅ alkyl radicals.

24. A process for cosmetic treatment of hair, comprising applying to the hair a cosmetic composition comprising, in a cosmetically acceptable medium, at least one polymer having a non-silicone backbone, comprising at least two nonphotoactivable, reactive chemical functional groups, which may be identical or different, wherein:
(i) a film obtained by drying the composition at room temperature (22±2° C.) and at a relative humidity level of 50%±5%, has a Young's modulus ranging from 1 to 100 MPa, measured at a thickness of 0.5 mm and at a pulling rate of 20 mm/min;
(ii) the at least two nonphotoactivable reactive chemical functional groups are chosen from the following monovalent and divalent groups:
epoxy,
anhydride,
acid chloride,
ethyleneimino,
aldehyde,
acetal and hemiacetal,
aminal and hemiaminal,
ketone, alpha-haloketone and alpha-hydroxyketone,
lactone and thiolactone,
isocyanate,
thiocyanate,
N-hydroxysuccinimide ester,
imide,
imine,
imidate,
oxazoline, oxazolinium, oxazine and oxazinium,
pyridylthiol,
thiosulphate,
acetoalkylate corresponding to the formula:
—OCO—A′—COCH₃,
wherein A′ is chosen from a bond and linear and branched alkylene groups comprising from 1 to 5 carbon atoms,
AX, and
ASO₂X,
wherein A is a group chosen from alkylene, arylene and aralkylene groups comprising from 1 to 22 carbon atoms, which may be optionally interrupted by at least one unsaturated ring, and may optionally comprise at least one hetero atom,
X is a leaving group chosen from halogens, OSO₃H, OSO₂CH3, OSO₂C₂H₅, OSO₂Tos, N(CH3)₃, OPO₃R₂, and CN,
wherein Tos is a tosylate group, and
R is chosen from a hydrogen atom and C₁ to C₅ alkyl radicals.

25. The process according to claim 24, wherein, before the application of the cosmetic composition to the hair, at least one other composition chosen from care, coloring, permanent waving, hair-makeup, hairstyle fixing and hairstyle-hold compositions is applied to the hair.

26. A method of forming a soft coating on hair, comprising applying to the hair a cosmetic composition comprising, in a cosmetically acceptable medium, at least one polymer having a non-silicone backbone, comprising at least two nonphotoactivable, reactive chemical functional groups, which may be identical or different, wherein:
(i) a film obtained by drying the composition at room temperature (22±2° C.) and at a relative humidity level of 50%±5%, has a Young's modulus ranging from 1 to 100 MPa, measured at a thickness of 0.5 mm and at a pulling rate of 20 mm/min;
(ii) the at least two nonphotoactivable reactive chemical functional groups are chosen from the following monovalent and divalent groups:
epoxy,
anhydride,
acid chloride,
ethyleneimino,
aldehyde,
acetal and hemiacetal,
aminal and hemiaminal,
ketone, alpha-haloketone and alpha-hydroxyketone,
lactone and thiolactone,
isocyanate,
thiocyanate,
N-hydroxysuccinimide ester,
imide,
imine,
imidate,
oxazoline, oxazolinium, oxazine and oxazinium,
pyridylthiol,
thiosulphate,
acetoalkylate corresponding to the formula:
—OCO—A′—COCH₃,
wherein A′ is chosen from a bond and linear and branched alkylene groups comprising from 1 to 5 carbon atoms,
AX, and
ASO₂X,
wherein A is a group chosen from alkylene, arylene and aralkylene groups comprising from 1 to 22 carbon atoms, which may be optionally interrupted by at least one unsaturated ring, and may optionally comprise at least one hetero atom,
X is a leaving group chosen from halogens, OSO₃H, OSO₂CH3, OSO₂C₂H₅, OSO₂Tos, N(CH3)₃, OPO₃R₂, and CN,
wherein Tos is a tosylate group, and
R is chosen from a hydrogen atom and C₁ to C₅ alkyl radicals.

27. A cosmetic composition for forming a soft coating on hair, comprising applying to the hair a cosmetic composition comprising, in a cosmetically acceptable medium, at least one polymer having a non-silicone backbone, comprising at least two nonphotoactivable, reactive chemical functional groups, which may be identical or different, wherein:
(i) a film obtained by drying the composition at room temperature (22±2° C.) and at a relative humidity level of 50%±5%, has a Young's modulus ranging from 1 to 100 MPa, measured at a thickness of 0.5 mm and at a pulling rate of 20 mm/min;
(ii) the at least two nonphotoactivable reactive chemical functional groups are chosen from the following monovalent and divalent groups:
epoxy,
anhydride,
acid chloride,
ethyleneimino,
aldehyde,
acetal and hemiacetal,
aminal and hemiaminal,
ketone, alpha-haloketone and alpha-hydroxyketone,
lactone and thiolactone,
isocyanate,
thiocyanate,
N-hydroxysuccinimide ester,
imide,
imine,
imidate,
oxazoline, oxazolinium, oxazine and oxazinium,
pyridylthiol,
thiosulphate,
acetoalkylate corresponding to the formula:
—OCO—A′—COCH₃,
wherein A′ is chosen from a bond and linear and branched alkylene groups comprising from 1 to 5 carbon atoms,
AX, and
ASO₂X,
wherein A is a group chosen from alkylene, arylene and aralkylene groups comprising from 1 to 22 carbon atoms, which may be optionally interrupted by at least one unsaturated ring, and may optionally comprise at least one hetero atom,
X is a leaving group chosen from halogens, OSO₃H, OSO₂CH3, OSO₂C₂H₅, OSO₂Tos, N(CH3)₃, OPO₃R₂, and CN,
wherein Tos is a tosylate group, and
R is chosen from a hydrogen atom and C₁ to C₅ alkyl radicals;
wherein said composition is effective to form a soft coating on the hair.
